(19) United States
(12) Patent Application Publication
(10) Pub. No.: US 2016/0341577 A1
(43) Pub. Date: Nov. 24, 2016

(54) Data Logger

(71) Applicant: WTW WISSENSCHAFTLICH-TECHNISCHE WERKSTÄTTEN GMBH, Weilheim (DE)

(72) Inventor: Michael Baumann, Neuburg (DE)

(21) Appl. No.: 15/114,587

(22) PCT Filed: Jan. 23, 2015
(86) PCT No.: PCT/EP2015/051322

§ 371 Date: Jul. 27, 2016

(30) Foreign Application Priority Data
Jan. 27, 2014 (DE) 10 2014 100 920.7

(51) Int. Cl. G01D 9/00; G01N 33/00; G01K 1/02

(52) U.S. Cl. CPC G01D 9/005; G01K 1/022; G01N 33/0036

(57) ABSTRACT

The invention relates to a data logger (1) for recording, storing, evaluating, displaying and/or reading measurement data with at least the following functional units, preferably arranged in a common housing: at least one sensor unit (2) for measuring environmental parameters in the form of analog measurement values, such as, in particular, temperature and/or humidity, at least one analog/digital converter (3) for converting the analog measurement values into digital measurement data, at least one processor unit (4) for generating at least one file (10, 11) from the digital measurement data, possibly in prepared form, at least one storage unit (5) for storing digital data, in particular the at least one file (10, 11), and at least one interface (6), preferably a USB interface, for transmitting at least digital data to and/or from the data logger (1), and a power source (7). In accordance with the invention, it is proposed that the processor unit (4) is also formed for generating an executable file (12) or that such executable file (12) is already stored in the data logger (1), whereas this executable file (12) is displayed upon connecting to an external computer (20) as a file (12) executable with the operating system (21) of the external computer (20) or is automatically executed by this operating system (21).

The present invention relates to a data logger in accordance with the preamble of claim 1.

A data logger conforming to this type for recording, evaluating and displaying data generated from measured values, such as temperature readings, is known from DE 10 2011 054 838 A1. Such data logger features a sensor unit, a storage unit and a processor unit, which are connected to the other units for recording measured values and for evaluating, displaying and storing data, whereas all device units are arranged in a common housing. A USB interface is provided on the housing for digital data exchange with an external computer.

Such data loggers are used in particular where a long-term recording of measurement parameters is to be undertaken (for example, in the monitoring of transport), whereas temperature and humidity data, as well as acceleration and/or vibration data, may be of particular interest. Moreover, such data may be used for monitoring the conditions in warehouses (temperature, humidity), for error analysis of systems, for monitoring and alerting during production processes and upon quality trials, and upon the recording of data in the hobby sector, such as the acquisition of flight data of a model airplane.

Accordingly, with a data logger, the various measurement data can be recorded and stored over a longer period of time, depending on the sensor system. If necessary, such data can be further processed, graphically displayed and edited for documentation.

A common use of data loggers is that, in particular, such a data logger is programmed at the place of consignment of a container or another means of transport and is set for the transported goods. At the destination, the data regarding temperature and/or humidity, etc. stored in the data logger during transport are then evaluated and analyzed for errors. Frequently, the corresponding files must be sent to the customer of the forwarding agency by e-mail, in order to verify that the goods have reached their destination without incident (for example, an excessive temperature increase for refrigerated goods, such as pharmaceuticals or frozen foods).

Both the direct reading of measurement data and the programming and configuration of the data logger by means of special software (for example, for the measurement of the measuring cycle and/or the permitted temperature limits) are carried out when the data logger is connected to the computer. For this purpose, for example with the data logger according to DE 10 2011 054 838 A1, the USB interface of the logger is formed as a multi-class interface, which registers the data logger upon connecting to the external computer, at the same time with at least two interface classes. Thus, the data logger of the computer is essentially synchronously recognizable for particular evaluation and/or programming software as a storage device by means of the first interface class and as a communication device by means of the second interface.

Typically, known data loggers provide the recorded and stored data in a PDF document. Of course, any other standard format is conceivable. Thereby, when connected to an external computer, under which, within the framework of this invention, all data processing systems are to be generally subsumed (including portable devices and smart phones, etc.), the data logger registers as the specified mass storage device. Thus, the user can recognize the data logger on the external computer as a disk drive on which the measurement data are stored in the corresponding format, for example, in PDF format. For transmitting the data stored in the data logger data to a recipient (forwarding agency headquarters, insurance company, client, etc.), the corresponding data may be attached to an e-mail to be generated by the operator at the destination, or special company software is stored on the external computer that automatically carries out the sending of the data. Alternatively, the data may be copied into the corresponding directories of the corporate network (intranet) or in an Internet storage location (the cloud).

The task of the present invention is to provide a data logger with increased operating comfort.

The task is solved with a data logger with the characteristics of the independent claim 1.

The advantages of the invention can be seen in particular in that—in accordance with a first alternative—the processor unit is not only formed for the storage of digital data in the storage unit, but may also generate an executable file (especially as an .EXE file). In accordance with a second alternative, such an executable file is already stored in the data logger, whereas the transfer of this file preferably takes place upon the programming of the data logger (measurement cycle, temperature range, alert limit, etc.). If the data logger in accordance with the invention is used to monitor the environmental conditions during the transport of goods (such as pharmaceuticals), the specified executable file can be executed with standard software when connecting to an external computer, typically at the destination that such executable file displays. Such standard software is always present, for example, in a computer with a Microsoft operating system. The display of the executable file on a screen connected to the external computer is carried out, for example, by the data logger being automatically displayed as a disk drive when connected to the computer, which displays the files found on the data logger by means of clicking on them, among which the specified executable file is located. If the data logger is not automatically displayed as a disk drive, the file structure is to be opened (with a Microsoft operating system, by means of Explorer), whereupon the disk drive of the data logger is displayed.

In accordance with an advantageous embodiment of the invention—in conjunction with an e-mail client (such as Microsoft Outlook) installed on the external computer—when executing the executable file, known as clicking on the file, an e-mail is generated on the external computer, and is preferably displayed on the screen. With this e-mail, at least one of the following actions is carried out automatically, that is, by executing the program by clicking on it:
insertion of a recipient's address in the e-mail,
insertion of a return address in the e-mail,
insertion of a subject in the e-mail,
insertion of content in the e-mail, and/or
attachment of a file generated by the processor unit from the data stored in the storage unit.

The information necessary for insertion (recipient address, sender address, subject, content), is fetched by the executable program from the data stored in the data logger. Preferably, such data were previously stored in a corresponding storage location in the data logger, preferably when programming the logger at the starting location of the transport.

Alternatively or additionally, the executable file may be configured such that the files generated by the processor unit are copied automatically when executing the program in a defined directory of a computer, server or Internet storage location (for example, the cloud). Furthermore, it may advantageously be provided that the executable file is formed in such a manner that it initiates the creation and sending of an e-mail to a predefined recipient, with which the recipient is informed of the storage of at least one file from the specified storage unit of the data logger in the external storage medium.

The arrangement in accordance with the invention makes it possible that, even without special company software and/or without any special training of the operating personnel, a transmission of data from the logger that is extremely simple, not error-prone or error-prone to an essentially low degree, and thus secure, is ensured. Since company-specific software is not required, there is no need for a corresponding installation that would be carried out on a wide variety of geographic locations while encountering various conditions for the computers used there. Since it can never be guaranteed that such installations will succeed on the existing computers without error, or that the appropriate personnel will at all be available for such installations, it is highly advantageous if an installation of company-specific software can be entirely dispensed with.

Similarly, the cost of error is reduced by—in accordance with one of the preferred embodiments of the invention (see above)—a preferably fully automated e-mail creation being realized by clicking on and executing the executable file, including the filling in of e-mail information and/or attachment of the files containing the measurement data to such e-mail. The advantage here is that the error rate is significantly reduced, since an operator is able to make fewer entry errors or attach fewer incorrect (or even falsified) files.

In the most simple case, the degree of automation in accordance with the invention by automatically generating an e-mail with preferably all relevant information (sender, recipient, subject, content, attached files) requires only the connection of the data logger to a computer connected to the Internet (on which only one e-mail client must be installed, which may simply be a standard e-mail client), the opening of the data logger disk drive registered as a disk drive on the computer and clicking on the executable file located on this disk drive. In accordance with the most preferred embodiment of the invention, the file initiates the opening of the e-mail client on the external computer and subsequently the generation of an e-mail and the automatic filling in of the essential information in this e-mail, along with the automatic attachment of one or more files generated by the processor unit, which may relate to the digital measurement data and include additional data. At that point, the e-mail is to be actively sent solely by the operator, preferably on the Internet, but even this step could be automated. The recipient that receives the file(s) may then evaluate, analyze and archive them, and make them available to third parties (forwarding agency, customer, insurance company, etc.). The recipient address(es) in the e-mail may relate, for example, to a company address, and/or an address in the cloud.

Preferably, the processing unit is formed in such a manner that it is capable of storing digital data, in particular digital data in raw or processed form, in the storage unit in the form of a PDF file and in a second file format, for example, as an ED3 file. At least one, preferably both files, are preferably automatically attached to the specified e-mail when executing the specified executable file.

In accordance with an advantageous embodiment, the processor unit is formed in such a manner that, in the specified PDF file, it is capable of storing processed data, which include, for example in tabular or graphic form, the measurement data (such as T-measurement values) and the associated measuring times along with, if applicable, a corresponding graph. Moreover, other event data may be stored in the PDF file. This PDF file serves to illustrate the maintaining of or deviation from the conditions during transport, and may contain other data, such as information regarding the transported goods and the transport quantity, the transport period and the transport route, the forwarding agent, etc.

The aforementioned generation of a PDF file has the additional advantage that, after the plugging of the data logger into an external computer, the data can be read by means of freely available standard software (for example, the popular Adobe® Reader) and can be transferred to the external computer.

Alternatively, preferably in addition, the processing unit is formed in such a manner that it generates a separate file in a different format, for example, of the ED3-type, with digital or digitized data as raw data, whereas such file is also attached to the specified e-mail—preferably automatically by clicking on the executable file.

Particularly advantageously, the processor unit can be programmed or configured by means of special evaluation and/or programming software, in particular for storing the specified e-mail recipient's address, the specified e-mail sender's address, the specified e-mail subject and/or the specified e-mail content, in a storage location of the data logger. This storage location may be, for example, part of the specified storage unit.

Even with the additional variants of the invention of the automatic storage or copying of the at least one file from the specified storage unit of the data logger in an external storage medium, it is advantageous if the address or the path of such specified external storage medium is stored in a storage location of the data logger. Moreover, this storage location may be part of the specified storage unit in the data logger.

In order to carry out an examination of the measurement result even without an external data processing system, it is advantageous if the data logger features an indicator, in particular a display, an operating element, in particular a key and/or a switch, and/or a combined indicator/operating unit, in particular a touchscreen.

Additional advantages of the invention are described in the following embodiment. The following is shown:

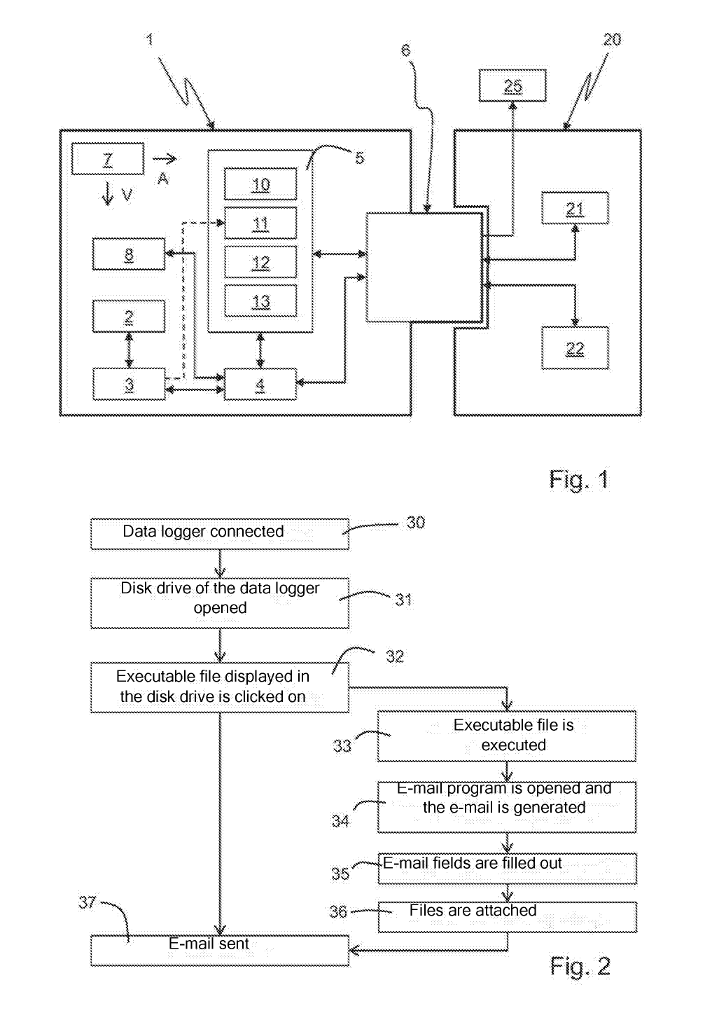

FIG. 1 a block diagram of the data logger, connected to an external computer, and

FIG. 2 a flow chart.

FIG. 1 shows a data logger 1, which is connected via an interface 6 to an external computer 20. The interface 6 is preferably formed as a USB interface, or as a firewire interface. For recording, storing, evaluating, displaying and/or reading data, the data logger 1 features a sensor unit 2, in this case formed in particular for recording the temperature, an analog/digital converter 3 for converting the measured analog data into digital data, a processor unit 4 and a storage unit 5. By means of the sensor unit 2, information, in particular the temperature, from the environment are continuously recorded and forwarded to the processor unit 4 over a long period of time. This prepares the measurement data in such a manner that they can be presented in particular graphically.

The raw data and/or the processed data are preferably stored by the processor unit 4 in the storage unit 5 as a PDF file 10. In the PDF file 10, the processed data (along with the corresponding time data) in tabular and/or graphic form and, if applicable, event data (battery change, operator intervention, transport route, freight, etc.) are included.

In an additional file 11 of the storage unit 5, the sensitive raw data the series of measurements are exclusively stored. Such raw data may also be stored in the storage unit 5 (see dashed line) as file 11, without passing through the processor unit 4. In addition, an optional timer, which records the associated date and time at each measurement signal and forwards such information (if applicable, through a buffer memory in which the measurement data can be temporarily stored) to the storage unit 5, is not shown. Alternatively, it is possible that the measurement data are always recorded in equal intervals, such that a timer is not necessary. The file for the raw data may in particular be of an ED3 format.

A power source 7, for example in the form of a block battery, supplies the different units of the data logger 1, in particular the sensor unit 2 and the processor unit 4, with the necessary energy.

In order to carry out an examination of the measurement result during or after the measurement, the data logger 1 also features an indicator/operating unit 8. This usually consists of a display, not shown here, and one or more switches or buttons. Further, the indicator/operating unit 8 may be formed as a touchscreen. Thus, a user may undertake an examination of the preferably graphically processed measurement data during or after the measurement. In addition, after the measurement, the prepared measurement data stored in the PDF file 10 may be viewed. Furthermore, there may be interactions through the indicator/operating unit 8, in order to, for example, start or stop the measurement.

Advantageously, the data logger 1 features a housing in which the specified units 2-8 are integrated. However, additional interfaces may be provided on the data logger 1, on which, for example, external sensor units can be connected. In principle, it is thereby possible that no sensor unit is provided in the housing.

FIG. 1 shows how the data logger 1 is connected to an external computer 20. This has an operating system 21, for example, a Microsoft Windows operating system. In addition, an e-mail client 22 (for example, Microsoft Outlook) is installed on the computer. The external computer 20 is also connected to the Internet.

In accordance with the invention, the data logger 1 is formed in such a manner that the processor unit 4 according to a first variant is capable of generating an executable file 12, which, in the present case, is also stored in the storage unit 5. According to an additional variant, such an executable file 12 is already stored therein, and need not be generated by the data logger 1, whereas storage in the storage unit 5 is also offered here; this executable file 12 may be stored in particular prior to the adding of the data logger 1 to a goods transport at the place of consignment by means of a computer in the storage unit 5 (not shown).

With reference to FIG. 2, a possible course of the transmission of files 10 and 11 to a destination address is described, so that the data stored in the storage unit 5 may be viewed or analyzed at a location other than the destination.

First, preferably at the destination of the transport of goods the data logger 1 that is also transported is connected through the interface 6 to the external computer 20 (step 30 in FIG. 2). Depending on the setting of the computer 20, the data logger 1 is automatically displayed as a newly attached disk drive on the screen connected to the computer 20, or such disk drive is manually placed for display on the screen (for example, using Explorer or the like). This displayed disk drive is opened by the user (step 31), whereupon the executable file 12 located on the disk drive is displayed and can be clicked on by the operator (step 32). By clicking on it, the executable file 12 is executed by the operating system 21 of the external computer 20 (step 33). The execution of the file 12 starts—if it has not already occurred—the e-mail client 22. In addition, an e-mail to this e-mail client 22 is generated (step 34), the fields of which (recipient and sender addresses, subject, content) are automatically filled in (step 35). For this purpose, the e-mail client 22 automatically accesses a storage location 13 of the storage unit 5 of the data logger 5, in which such information has been stored, expediently through programming the data logger 1 at the place of consignment of the goods transport. Such programming preferably takes place together with the programming of the data logger 1 with respect to measuring cycle, measurement limits, etc., which is carried out through the interface 6. In addition, for the delivery of the specified information in the e-mail, the files 10, 11 are attached to this e-mail (step 36).

At that point, the e-mail is still sent exclusively by the operator (step 37). The files 10, 11 are stored in this manner, for example, in web-based cloud software, and are available for viewing and processing. The e-mail server may also be provided to the forwarding agent, to the customer or to another party. The PDF file 10 serves, in particular, for the visualization of the transport parameters, while the file 11 reproduces the raw data of the measurements, in the present case, for example, in ED3 format.

According to the example discussed, the steps shown in the left half of FIG. 2 are to be carried out by the operator, while the steps in the right half run automatically. However, it is also readily possible that additional steps (if applicable, even all of the steps up to the connection of the data logger 1 to the external computer 20) run automatically.

In the figures, only the case of a single sensor unit is shown. Multiple sensor units with multiple analog/digital converters may be readily provided.

In accordance with an alternative or additional embodiment, by clicking on the executable file 12, the automatic transmission of the files 10, 11 from the specified storage unit 5 to an external storage medium 25 (see FIG. 1) is initiated. This defined directory may be present in a computer, a server or an Internet location (for example, the cloud). Moreover, the executable file 12 may be formed in such a manner that it initiates the creation and sending of an e-mail to a defined recipient (for example, stored in storage location 13), with which it is informed of the storage of the files 10, 11 in the specified external storage medium 25. The aforementioned storage or copying of files 10, 11 in the external storage medium 25 and all other steps, including e-mail notification, may take place automatically, if only the data logger 1 is connected to the external computer 20.

The invention may be used both for one-time and for reusable data loggers.

The present invention is not limited to the presented and described embodiment. Variations within the framework of the patent claims, such as a combination of features, even if they are described in different embodiments. For example, it may be provided that the PDF file 10 is automatically generated only upon connecting to the data logger 1 to the external computer 20.

### LIST OF REFERENCE SIGNS

1 Data logger
2 Sensor unit
3 Analog/digital converter
4 Processor unit
5 Storage unit
6 Interface
7 Power source
8 Indicator/operating unit
10 PDF file
11 ED3 file
12 Executable file
13 Storage location
20 External computer
21 Operating system
22 E-mail client
25 External storage medium
30-36 Sequence steps

1. Data logger (1) for recording, storing, evaluating, displaying and/or reading measurement data with at least the following functional units, preferably arranged in a common housing:
at least one sensor unit (2) for measuring environmental parameters in the form of analog measurement values, such as, in particular, temperature and/or humidity,
at least one analog/digital converter (3) for converting the analog measurement values into digital measurement data,
at least one processor unit (4) for generating at least one file (10, 11) from the digital measurement data, possibly in prepared form,
at least one storage unit (5) for storing digital data, in particular the at least one file (10, 11), and
at least one interface (6), preferably a USB interface, for transmitting at least digital data to and/or from the data logger (1), and
a power source (7),
characterized in that,
the processor unit (4) is also formed for generating an executable file (12) or that such executable file (12) is already stored in the data logger (1),
whereas this executable file (12) is displayed upon connecting to an external computer (20) as a file (12) executable with the operating system (21) of the external computer (20) or is automatically executed by this operating system (21).

2-9. (canceled)
